(19) United States
(12) Patent Application Publication
(10) Pub. No.: US 2025/0107809 A1
(43) Pub. Date: Apr. 3, 2025

(54) SAFE MODE AND FIRE MODE FOR ROBOTIC CLIP APPLIER

(71) Applicant: AURIS HEALTH, INC., Santa Clara, CA (US)

(72) Inventors: Joshua L. Godfrey, Santa Clara, CA (US); Henry A. PECK, San Francisco, CA (US); Alexander T. HASSAN, San Francisco, CA (US)

(21) Appl. No.: 18/979,462

(22) Filed: Dec. 12, 2024

Related U.S. Application Data
(63) Continuation of application No. PCT/IB2023/056736, filed on Jun. 29, 2023.
(60) Provisional application No. 63/356,687, filed on Jun. 29, 2022.

(51) Int. Cl. A61B 17/128; A61B 17/00; A61B 17/122; A61B 34/20; A61B 34/30; A61B 34/37

(52) U.S. Cl. CPC A61B 17/1285; A61B 17/122; A61B 34/20; A61B 34/37; A61B 2017/00946; A61B 2017/00973; A61B 2034/302

(57) ABSTRACT

An apparatus includes an instrument, a user input, and a control module. The instrument includes an end effector with first and second jaws that are operable to transition between a fully open position and a fully closed position. The control module is in communication with the instrument and the first user input. The control module is configured to transition between a safe operating mode and a fire operating mode. In the safe operating mode, the control module is configured to prevent or restrict closure of the first and second jaws in response to an activation signal from the first user input. In the fire operating mode, the control module is configured to generate a power drive signal that drives closure of the first and second jaws to the fully closed position.

### PRIORITY

This application is a continuation of International Patent Application No. PCT/IB2023/056736, filed Jun. 29, 2023, entitled “SAFE MODE AND FIRE MODE FOR ROBOTIC CLIP APPLIER,” which claims priority to U.S. Provisional Patent Application No. 63/356,687, entitled “SAFE MODE AND FIRE MODE FOR ROBOTIC CLIP APPLIER,” filed Jun. 29, 2022. The contents of all of the above-referenced patent applications are hereby incorporated by reference in their entirety for all purposes.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probes, etc. Each of these structures performs functions for the surgeon, for example, cutting tissue, coagulating tissue, holding or driving a needle, grasping a blood vessel, dissecting tissue, or cauterizing tissue.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first example of a robotic system configured for a laparoscopic procedure;

FIG. 2 depicts a perspective view of a second example of a robotic system;

FIG. 3 depicts an end elevational view of the robotic system of FIG. 2;

FIG. 4 depicts the end elevational view of the robotic system of FIG. 2 including an example of a pair of robotic arms;

FIG. 5 depicts a partially exploded perspective view of the robotic arm of FIG. 4 having an instrument driver and an example of a surgical instrument;

FIG. 6A depicts a side elevational view of the surgical instrument of FIG. 5 in a retracted position;

FIG. 6B depicts a side elevational view the surgical instrument of FIG. 5 in an extended position;

FIG. 7A depicts a top plan view of an example of an end effector that may be incorporated into the surgical instrument of FIG. 5, with the end effector in an open state;

FIG. 7B depicts a top plan view of the end effector of FIG. 7A in a partially closed state;

FIG. 7C depicts a top plan view of the end effector of FIG. 7A in a fully closed state, with an example of a clip disposed in the end effector;

FIG. 8 depicts a perspective view of an example of a control console that may be used with the robotic systems of FIGS. 1 and 2;

FIG. 9A depicts a perspective view of a hand-activated user input assembly of the control console of FIG. 8, with pivoting arms of the user input assembly in a first position;

FIG. 9B depicts a perspective view of the hand-activated user input assembly of FIG. 9A, with the pivoting arms in a second position;

FIG. 10 depicts a plan view of an example of a display that may be rendered via the control console of FIG. 8;

FIG. 11 depicts a graph showing an example of a correlation between an end effector closure state and a user input activation state, based on different operating modes, as may be employed in the robotic systems of FIGS. 1 and 2;

FIG. 12 depicts a flow chart showing an example of a control algorithm that may be executed via the control console of FIG. 8.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved case of use such that one or more of the instruments of the robotically-enabled medical system may be controlled by a single operator.

### I. EXAMPLE OF ROBOTICALLY-ENABLED MEDICAL SYSTEM

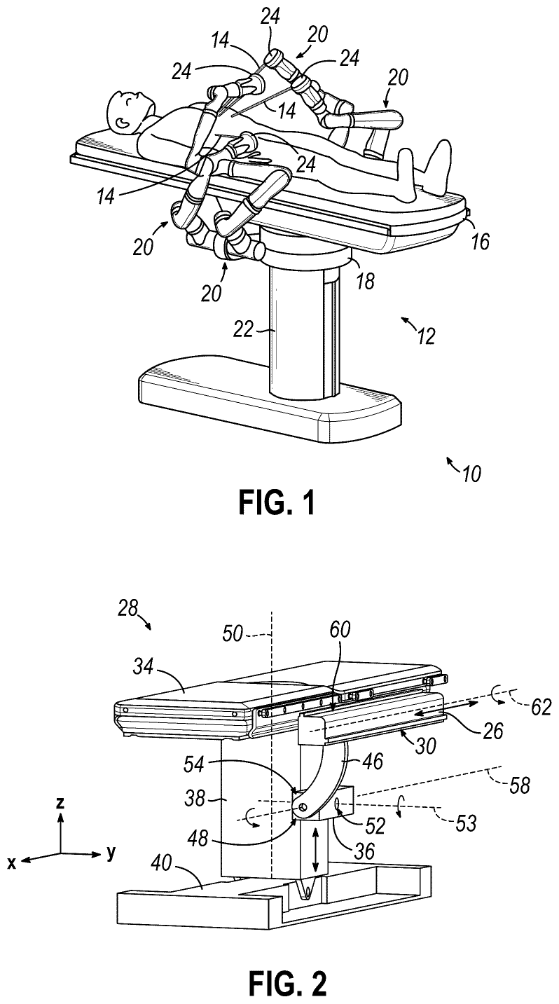

FIG. 1 shows an example of a robotically-enabled medical system, including a first example of a robotic system (10). Robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of robotic system (10) may be constructed and operable in accordance with at least some of the teachings of any of the various patents, patent application publications, and patent applications that are cited herein.

### A. Example of Robotic System with Annular Carriage

As shown in FIG. 1, robotic system (10) includes table system (12) having a platform, such as a table (16), with a plurality of carriages (18) which may also be referred to herein as “arm supports,” respectively supporting the deployment of a plurality of robotic arms (20). Robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14), into different access points on the patient. In alternative examples, such as those discussed below in greater detail, robotic system (10) may include a surgical bed with adjustable arm supports including a bar (26) (see FIG. 2) extending alongside. One or more robotic arms (20) may be attached to carriages (18) (e.g., via a shoulder with an elbow joint). Robotic arms (20) are vertically adjustable so as to be stowed compactly beneath table (16), and subsequently raised during use.

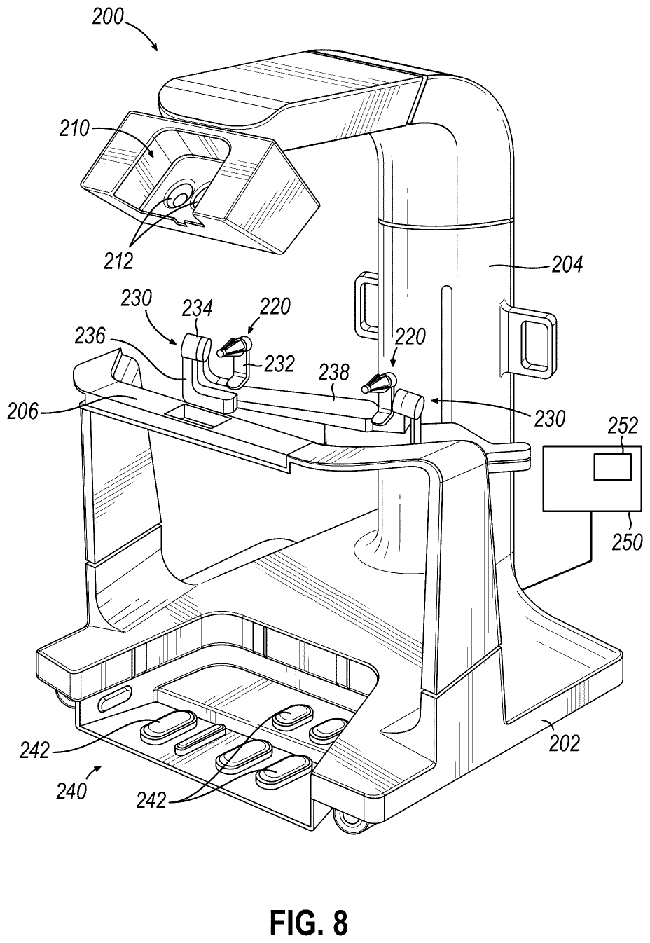

Robotic system (10) may also include a tower (not shown) that divides the functionality of robotic system (10) between table (16) and the tower to reduce the form factor and bulk of table (16). To this end, the tower may provide a variety of support functionalities to table (16), such as computing and control capabilities, power, fluidics, optical processing, and/or sensor data processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In some versions, the tower may include gas tanks to be used for insufflation. Examples of forms that may be taken by a tower and/or console are described in greater detail below with reference to FIGS. 8-10.

### B. Example of Robotic System with Bar Carriage

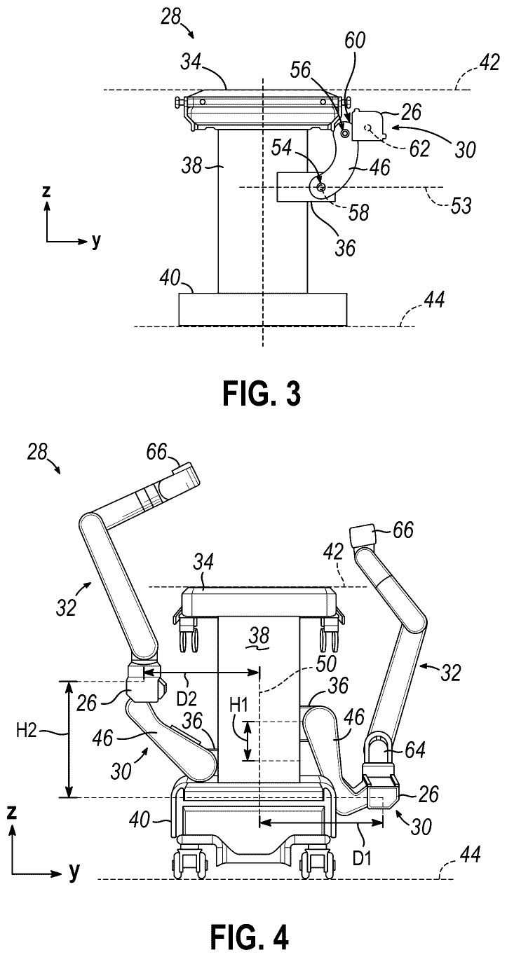

FIGS. 2-4 show another example of a robotic system (28). Robotic system (28) of this example includes one or more adjustable arm supports (30) including bars (26) that are configured to support one or more robotic arms (32) relative to a table (34). In the present example, a single adjustable arm support (30) (FIGS. 2-3) and a pair of adjustable arm supports (30) (FIG. 4) are shown, though additional arm supports (30) may be provided about table (34). Each adjustable arm support (30) is configured to selectively move relative to table (34) so as to alter the position of adjustable arm support (30), and/or any robotic arms (32) mounted thereto, relative to table (34) as desired. Such adjustable arm supports (30) may provide high versatility to robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 2-4, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36). Vertical carriage (36) is configured to move up or down along or relative to a column (38) and a base (40), both of which support table (34). A second degree of freedom allows adjustable arm support (30) to tilt about an axis extending in the y-direction. For example, adjustable arm support (30) includes a rotary joint, which allows adjustable arm support (30) to align with table (34) when table (34) is in a Trendelenburg position or other inclined position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) together support table (34) relative to a support surface, which is shown along a support axis (42) above a floor axis (44) in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) mechanically constrains third joint (54) to maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

FIG. 4 shows a version of robotic system (28) with two adjustable arm supports (30) mounted on opposite sides of table (34). A first robotic arm (32) is attached to one such bar (26) of first adjustable arm support (30). This first robotic arm (32) includes a connecting portion (64) attached to a first bar (26). Similarly, a second robotic arm (32) includes connecting portion (64) attached to the other bar (26). As shown in FIG. 4, vertical carriages (36) are separated by a first height (H1), and bar (26) is disposed a second height (H2) from base (40). The first bar (26) is disposed a first distance (D1) from vertical axis (50), and the other bar (26) is disposed a second distance (D2) from vertical axis (50). Distal ends of first and second robotic arms (32) respectively include instrument drivers (66), which are configured to attach to one or more instruments such as those discussed below in greater detail.

In some versions, one or more of robotic arms (32) has seven or more degrees of freedom. In some other versions, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and connecting portion (64) (1-degree of freedom including translation). In some versions, the insertion degree of freedom is provided by robotic arm (32); while in some other versions, an instrument such as surgical instrument includes an instrument-based insertion architecture.

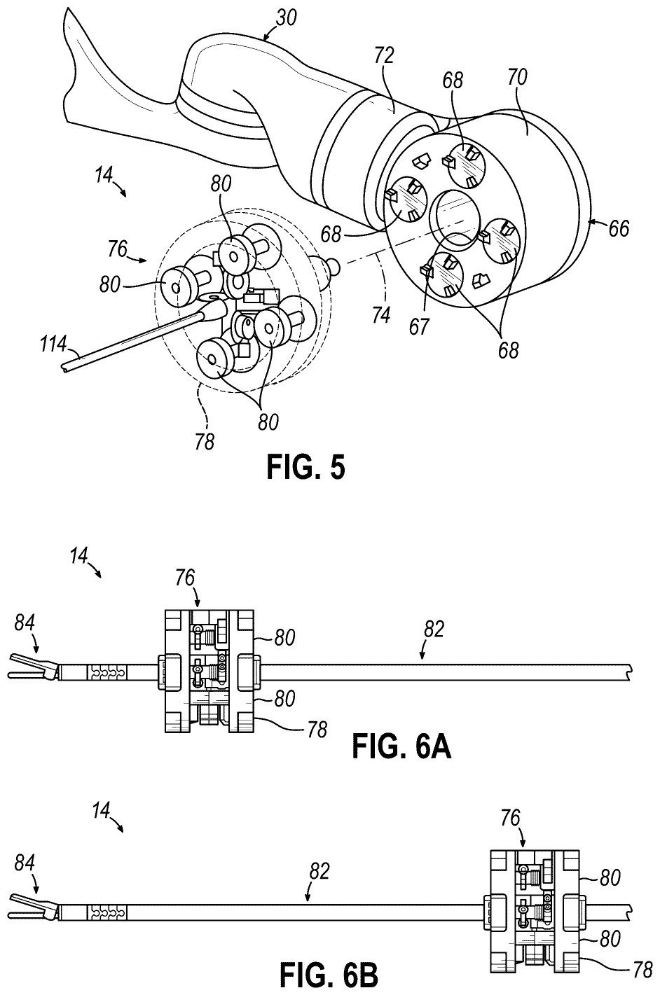

FIG. 5 shows one example of instrument driver (66) in greater detail, with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be configured to be detached, removed, and interchanged from instrument driver (66) for individual sterilization or disposal by the medical professional or associated staff. In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

### C. Example of Surgical Instrument with Instrument-based Insertion Architecture

FIGS. 5-6B show surgical instrument (14) having the instrument-based insertion architecture as discussed above. Surgical instrument (14) includes elongated shaft assembly (82), end effector (84) connected to and extending distally from shaft assembly (82), and instrument base (76) coupled to shaft assembly (82). Insertion of shaft assembly (82) is grounded at instrument base (76) such that end effector (84) is configured to selectively move longitudinally from a retracted position (FIG. 6A) to an extended position (FIG. 6B), vice versa, and any desired longitudinal position therebetween. As used herein, the retracted position is shown in FIG. 6A and places end effector (84) relatively close and proximally toward instrument base (76); whereas the extended position is shown in FIG. 6B and places end effector (84) relatively far and distally away from instrument base (76). Insertion into and withdrawal of end effector (84) relative to the patient may thus be facilitated by surgical instrument (14), although it will be appreciated that such insertion into and withdrawal may also occur via adjustable arm supports (30) in one or more examples.

As shown in FIGS. 5-6B, and in cooperation with instrument driver (66) discussed above, surgical instrument (14) includes an elongated shaft assembly (82) and an instrument base (76) with an attachment interface (78) having a plurality of drive inputs (80) configured to respectively couple with corresponding drive outputs (68). Shaft assembly (82) of instrument (14) extends from a center of instrument base (76) with an axis substantially parallel to the axes of the drive inputs (80) as discussed briefly above. With shaft assembly (82) positioned at the center of instrument base (76), shaft assembly (82) is coaxial with instrument driver axis (74) when attached and movably received in clearance bore (67). Thus, rotation of rotational assembly (70) causes shaft assembly (82) of surgical instrument (14) to rotate about its own longitudinal axis while clearance bore (67) provides space for translation of shaft assembly (82) during use.

The foregoing examples of surgical instrument (14) and instrument driver (66) are merely illustrative examples. Robotic arms (32) may interface with different kinds of instruments in any other suitable fashion using any other suitable kinds of interface features. Similarly, different kinds of instruments may be used with robotic arms (32), and such alternative instruments may be configured and operable differently from surgical instrument (14).

### D. Example of Clip Applier End Effector

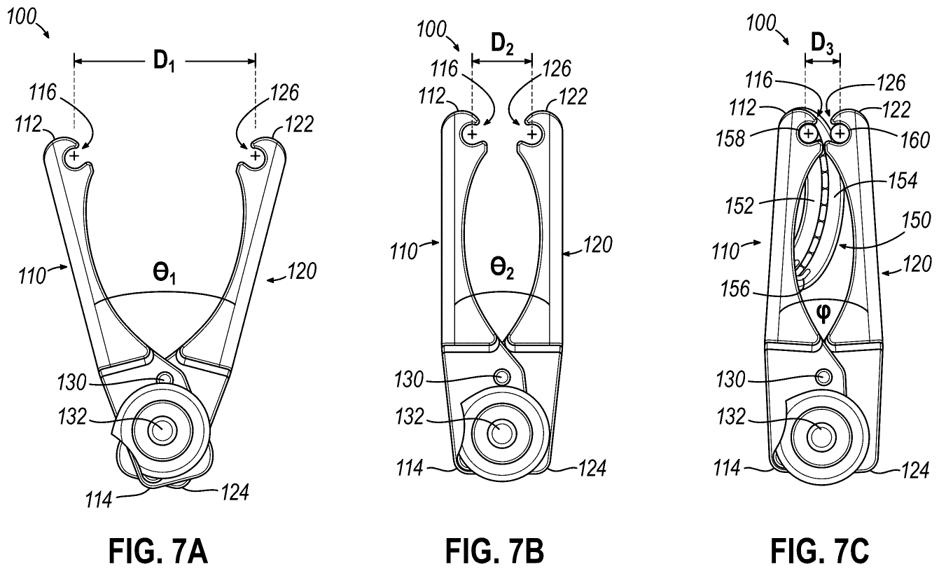

FIGS. 7A-7C show an example of an end effector (100), which represents a form that may be taken by end effector (84) of surgical instrument (14). End effector (100) of this example includes a pair of jaws (110, 120) that are coupled together at a pivot (130). Each jaw (110, 120) includes a respective distal end (112, 122), which move toward or away from each other based on a closure state of end effector (84). Each distal end (112, 122) includes a respective distal engagement feature (116, 126). In the present example, each distal engagement feature (116, 126) is in the form of a notch that is configured to receive a respective pin (158, 160) of a ligation clip (150).

An example of ligation clip (150) is shown in FIG. 7C. As shown, ligation clip (150) of this example includes a pair of arms (152, 154) that are joined together at a proximal hinge (156). Pins (158, 160) are at distal regions of arms (152, 154), such that jaws (110, 120) are operable to drive ligation clip (150) from an open state (not shown) to a closed state (FIG. 7C) by urging pins (158, 160) toward each other. In some versions, ligation clip (150) includes a latching feature that is configured to maintain ligation clip (150) in a closed state once ligation clip (150) reaches the closed state. In addition, or in the alternative, ligation clip (150) may include one or more malleable features that is/are configured to maintain ligation clip (150) in a fully closed state. In some such versions, the one or more malleable features is/are also configured to maintain ligation clip (150) in a fully open state, and/or in a partially closed state, until sufficient force is applied to ligation clip (150) to overcome the malleability.

End effector (100) further includes an actuator (132) that is operable to drive jaws (110, 120) to transition between a fully open state (FIG. 7A) and a fully closed state (FIG. 7C). Actuator (132) is positioned at proximal ends (114, 124) of jaws (110, 120). By way of example only, actuator (132) and jaws (110, 124) may include complementary camming features that are configured to drive pivotal movement of jaws (110, 120) about pivot (130) as actuator (132) moves relative to jaws (110, 120). By way of further example only, such driving movement of actuator (132) relative to jaws (110, 120) may include rotational movement, linear movement, and/or other kinds of movement. Actuator (132) may include one or more components operatively coupled with one or more corresponding components in instrument base (76) and shaft assembly (82) such that actuator (132) may ultimately be driven by instrument driver (24, 66) of robotic arm (20, 32).

FIG. 7A shows end effector (84) in a fully open state, where distal engagement features (116, 126) are separated from each other by a first distance (D1); and where jaws (110, 120) cooperate to define a first angle (Θ1), with the vertex of first angle (Θ1) being proximal to pivot (130). In the present example, when end effector (84) is in the fully open state, and ligation clip (150) is disposed in end effector (84), arms (152, 154) of ligation clip (150) have sufficient separation to receive a tissue structure (e.g., vessel, etc.) between arms (152, 154). In some scenarios, arms (152, 154) of ligation clip (150) may still have sufficient separation to receive a tissue structure (e.g., vessel, etc.) between arms (152, 154) even when end effector (84) is in a state of partial closure.

FIG. 7B shows end effector (84) in a partially closed state, where distal engagement features (116, 126) are separated from each other by a second distance (D2); and where jaws (110, 120) cooperate to define a second angle (Θ2), with the vertex of second angle (Θ2) being proximal to pivot (130). Second distance (D2) is shorter than first distance (D1). In the example shown in FIG. 7B, second angle (Θ2) is approximately 0 degrees, such that jaws (110, 120) are parallel with each other. Alternatively, jaws (110, 120) may cooperate to define various other non-zero angles in different partially closed states. In some scenarios, arms (152, 154) of ligation clip (150) may still have sufficient separation to receive a tissue structure (e.g., vessel, etc.) between arms (152, 154) when end effector (84) is the state of partial closure shown in FIG. 7B.

FIG. 7C shows end effector end effector (84) in a fully closed state, where distal engagement features (116, 126) are separated from each other by a third distance (D3); and where jaws (110, 120) cooperate to define an angle (φ), with the vertex of angle (φ) being positioned distally in relation to distal ends (112, 122). In the present example, when end effector (84) is in the fully closed state, ligation clip (150) is also in a fully closed state. In some versions, after end effector (84) reaches the fully closed state shown in FIG. 7C, actuator (132) may continue to drive distal ends (112, 122) toward each other to thereby increase a closure pressure on ligation clip (150). This increasing closure pressure on ligation clip (150) may in turn increase the clamping pressure of ligation clip (150) on tissue disposed between arms (152, 154) of ligation clip (150).

### II. EXAMPLE OF USER INTERFACE FEATURES

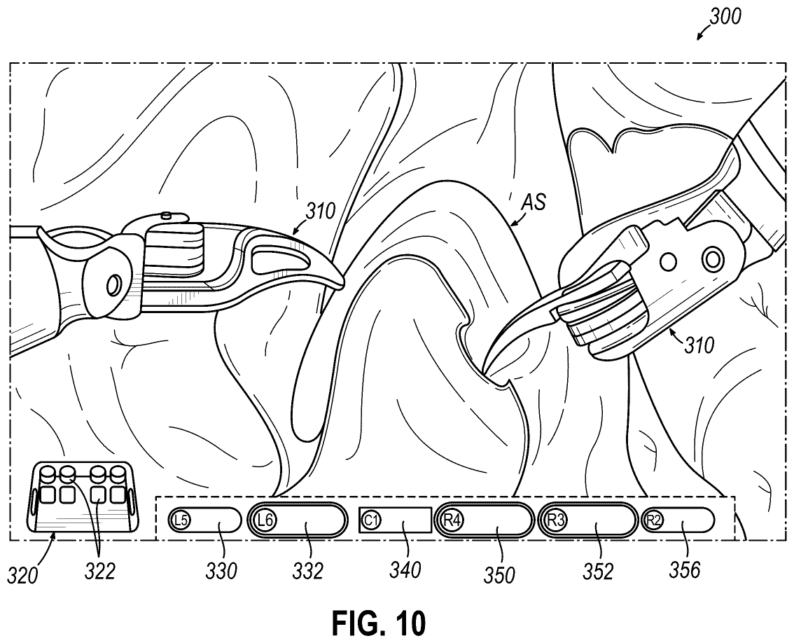

As noted above, a robotic system (10, 28) may include a tower and/or console with components that are operable to control operation of carriages (18), arm supports (30), bars (26), robotic arms (20, 32), table (16, 34), instrument drivers (24, 66), surgical instruments (14), and/or other components of robotic system (10, 28). FIG. 8 shows an example of such a console (200). Console (200) of the present example includes a base (202) and an upright member (204) extending from base (202). A viewing assembly (210) is supported by upright member (204) at a position where an operator may position their eyes at viewing windows (212) of upright member (204). Viewing assembly (210) may present a screen to the operator via viewing windows (212), with the screen displaying imagery such as that described in greater detail below with reference to FIG. 10.

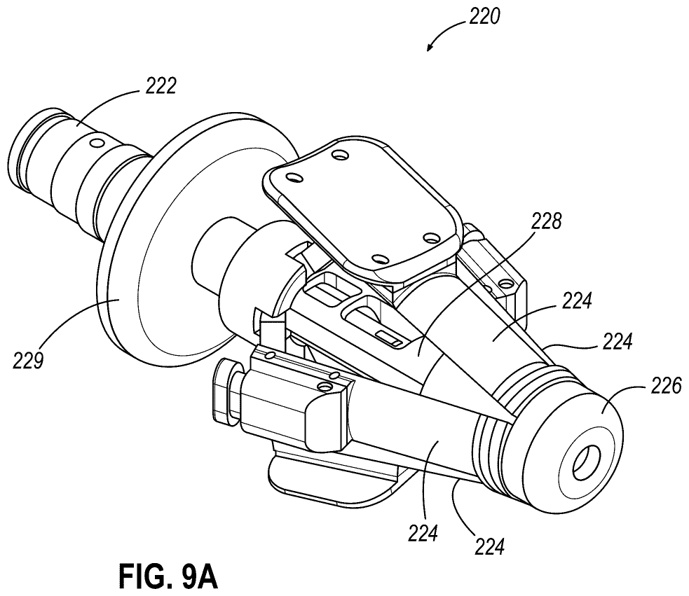

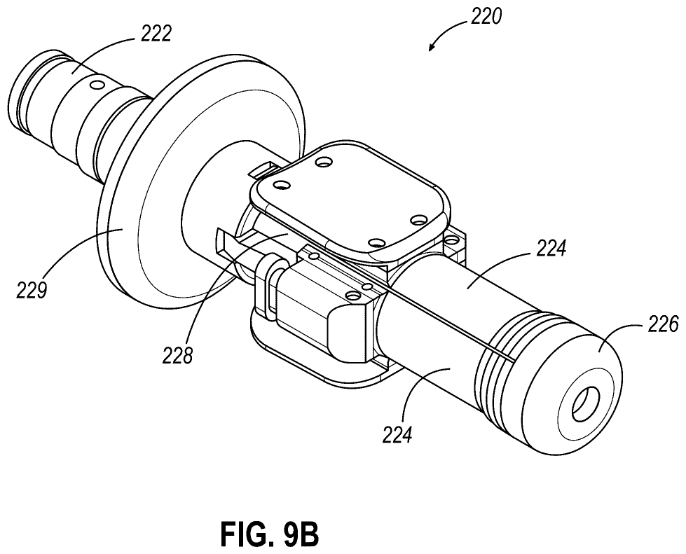

Console (200) also includes an arm rest (206) and a pair of user input assemblies (220) positioned near arm rest (206), such that an operator may rest their arms on arm rest (206) while manipulating user input assemblies (220) with their hands. Each user input assembly (220) is coupled with upright member (204) via an arm assembly (230), which is formed by a plurality of arm segments (232, 234, 236, 238) that are pivotably coupled to each other to allow substantial freedom of movement of user input assemblies (220) relative to upright member (204). In some versions, movement of user input assemblies (220) relative to upright member (204) provides corresponding movement of one or more robotic arms (20, 32) and/or certain features of robotic arms (20, 32). In addition, movement or other activation of certain components of user input assemblies (220) may provide corresponding movement or activation of features of instrument (14). Examples of such components of user input assemblies (220), and examples of corresponding movement or activation of features of instrument (14), will be described in greater detail below with reference to FIGS. 9A-9B.

Console (200) of the present example further includes a pedalboard (240) at the front of base (202). Pedalboard (240) includes a plurality of pedals (242) that may be actuated by an operator's foot. In some versions, one or more of pedals (242) includes a foot-activated switch that is operable to toggle between different states. For instance, some such versions of pedals (242) may be operable to selectively activate or deactivate an operational state of robotic system (10, 28). Similarly, some versions of pedals (242) may be operable to toggle among various operational modes of robotic system (10, 28). Some versions of pedals (242) may also be operable to selectively activate or deactivate a component of instrument (14). In addition to one or more of pedals (242) including a foot-activated switch, or as an alternative to such kinds of pedals (242), one or more pedals (242) may provide a variable control instead of a simple on-off/toggle type of control. Alternatively, pedals (242) may have any other suitable form and/or functionality as will be apparent to those skilled in the art in view of the teachings herein.

As also shown in FIG. 8, console (200) may be coupled with a tower (250), which is shown schematically in FIG. 8. Tower (250) may provide an interface between console (200) and the rest of robotic system (10, 28). For instance, tower (250) may include a processor (252) that is configured to receive control inputs received via user input assemblies (220) and pedalboard (240) and convert such inputs into operation of corresponding components of robotic system (10, 28) and instrument (14). For instance, processor (252) may convert user input signals into power drive signals. Processor (252) may also be operable to drive the display of a screen that is visible to the operator via viewing windows (212) of viewing assembly (210). Various components and functionalities that may be incorporated into tower (250) will be apparent to those skilled in the art in view of the teachings herein. In some variations, the components and functionalities that might otherwise be incorporated into tower (250) may be incorporated directly into console (200) and/or elsewhere, such that tower (250) need not necessarily be provided as a separate component between console (200) and the rest of robotic system (10, 28). For instance, processor (252) may be incorporated into console (200), into instrument (14), into table system (12), etc.

FIGS. 9A-9B show hand-activated user input assembly (220) in greater detail. As shown, hand-activated user input assembly (220) of the present example includes a plug (222) that is configured for insertion into a corresponding socket (not shown) of arm assembly (230). Some versions of arm assembly (230) may be configured to removably receive different types of user input features, such that the user input assembly (220) shown in FIGS. 9A-9B represents just one of many possible different modular configurations. User input assembly (220) of this example further includes a plurality of activation arms (224). The distal ends of activation arms (224) are pivotably coupled with a central shaft (228) of user input assembly (220) via a hub (226). With this configuration, the operator may grasp activation arms (224) with the operator's fingertips and thereby urge activation arms (224) from a first position (FIG. 9A) to a second position (FIG. 9B). In some versions, activation arms (224) include one or more resilient members (e.g., leaf springs, torsion springs, etc.) that may resiliently urge activation arms (224) toward the first position (FIG. 9A), such that activation arms (224) resiliently return to the first position (FIG. 9A) when the operator releases their grasp on activation arms (224) after reaching the second position (FIG. 9B) (or after reaching some intermediate position between the first and second positions).

As activation arms (224) transition from the first position (FIG. 9A) to the second position (FIG. 9B), user input assembly (220) generates an activation signal. In some versions, this activation signal is generated throughout the range of travel of activation arms (224) transition from the first position (FIG. 9A) to the second position (FIG. 9B), with a characteristic (e.g., amplitude, etc.) of the activation signal varying based on the degree to which activation arms (224) have transitioned from the first position (FIG. 9A) to the second position (FIG. 9B). In some other versions, the activation signal is not generated until activation arms reach the second position FIG. 9B). In either case, the activation signal generated by user input assembly (220) may be transmitted to processor (252), which may convert the activation signal from user input assembly (220) into a drive power drive signal that drives end effector (100), etc., in response to the activation signal from user input assembly (220), in accordance with the teachings herein. By way of further example only, user input assembly (220) may be configured and operable in accordance with at least some of the teachings of U.S. Pub. No. 2021/0298857, entitled “Hand-Manipulated Input Device with Hall Effect Sensor for Robotic System,” published Sep. 30, 2021, the disclosure of which is incorporated by reference herein, in its entirety.

In view of the foregoing, when an operator activates user input assembly (220) by driving activation arms (224) from the first position (FIG. 9A) toward the second position (FIG. 9B), such activation may provide corresponding actuation of one or more components of instrument (14). For instance, in some versions where instrument (14) includes an end effector like end effector (100) of FIGS. 7A-7C, jaws (110, 120) may close in proportion to the degree to which activation arms (224) have transitioned from the first position (FIG. 9A) to the second position (FIG. 9B). By way of further example, when activation arms (224) are in the first position as shown in FIG. 9A, jaws (110, 120) may be in the fully open state as shown in FIG. 7A. When activation arms (224) are in the second position as shown in FIG. 9B, jaws (110, 120) may be in the fully closed state as shown in FIG. 7C. When activation arms (224) are in an intermediate position (not shown) between the first position (FIG. 9A) and the second position (FIG. 9B), jaws (110, 120) may be in a partially closed state (e.g., as shown in FIG. 7B or somewhere else between the fully open state shown in FIG. 7A and the fully closed state shown in FIG. 7C). Thus, user input feature (220) may provide the operator with an intuitive control experience, where pivotal movement of jaws (110, 120) mimics pivotal movement of activation arms (224) to some degree.

User input assembly (220) of this example further includes a circular flange (229), which is fixedly secured to shaft (228). In some versions, the operator may grasp user input assembly (220) via flange (229) or hub (226) to thereby move the entirety of user input assembly (220) within three-dimensional space, with the joints between segments (232, 234, 236, 238) of arm assembly (230) accommodating such movement of the entirety of user input assembly (220) within three-dimensional space. In some versions, when the entire user input assembly (220) is moved within three-dimensional space to cause movement of one or more segments (232, 234, 236, 238) of arm assembly (230), this movement of one or more segments (232, 234, 236, 238) of arm assembly (230) may provide corresponding movement of one or more robotic arms (20, 32). In some such versions, user input assembly (220) is only operable to drive movement of components of surgical instrument (14) via instrument driver (66) when segments (232, 234, 236, 238) of arm assembly (230) remain stationary. In still other versions, at least a portion of a user input assembly (220) is further operable to cause movement of one or more robotic arms (20, 32), even if segments (232, 234, 236, 238) of arm assembly (230) remain stationary. As another variation, some or all of segments (232, 234, 236, 238) of arm assembly (230) may be omitted.

Once the operator has achieved the desired positioning and orientation of one or more robotic arms (20, 32), the operator may shift their engagement of user input assembly (220) from circular flange (229) or hub (226) to activation arms (224), to thereby control instrument (14) as described above. In some versions, one or more of pedals (242) may be operable to trigger an electrical lockout of arm assembly (230), thereby preventing further movement of robotic arms (20, 32) in response to subsequent movement of arm assembly (230). In other words, after the electrical lockout pedal (242) is activated, the operator may still be able to move arm assemblies (230) (e.g., to provide ergonomic comfort); yet such movement of arm assemblies (230) will not result in corresponding movement of robotic arms (20, 32).

FIG. 10 shows an example of a display (300) that may be rendered via viewing assembly (210) of console (200). As shown, display (300) of this example includes a real-time endoscopic view of end effectors (310) in relation to an anatomical structure (AS). End effectors (310) are at distal ends of respective instruments (14), which are coupled with respective robotic arms (20, 32). Display (300) further includes a graphical representation (320) of pedalboard (240), including graphical representations (322) of pedals (242). Graphical representation (320) is overlaid on a bottom corner of the real-time endoscopic image in this example, though graphical representation (320) may instead be positioned at any other suitable location. Graphical representation (320) may be used to display the real-time state of pedals (242). For instance, when an operator activates a pedal (242) with their foot, the corresponding graphical representation (322) of that pedal (242) may illuminate, change color, or provide some other form of visual feedback to indicate such activation of pedal (242). In some versions, that graphical representation (322) may remain illuminated, remain the changed color, etc., for the duration through which the operator keeps the corresponding pedal (242) depressed. In some other versions, such as those where a pedal (242) is used to toggle between two or more operational states, the operator may simply tap pedal (242) to toggle between the operational states, and the corresponding graphical representation (322) may remain illuminated, remain the changed color, etc., to indicate the toggled state even if the operator is no longer depressing pedal (242). In some variations, graphical representation (320) is omitted. In some versions where graphical representation (320) is omitted, indicators (330, 332, 334, 340, 350, 352, 356) may effectively convey the kind of information described above in connection with graphical representation (320).

Display (300) of the present example further includes an array of indicators (330, 332, 334, 340, 350, 352, 356) adjacent to graphical representation (320) of pedalboard (240). Indicators (330, 332, 334, 340, 350, 352, 356) are overlaid along the bottom of the real-time endoscopic image in this example, though indicators (330, 332, 334, 340, 350, 352, 356) may instead be positioned at any other suitable location. In the present example, each indicator (330, 332, 334, 340, 350, 352, 356) is associated with a corresponding robotic arm (20, 32). For instance, each indicator (330, 332, 334, 340, 350, 352, 356) may include a textual or graphical representation indicating the type of instrument (14) that is secured to the corresponding robotic arm (20, 32). In addition, to the extent that console (200) enables the operator to select which robotic arm (20, 32) may be controlled via each user input assembly (220), indicators (330, 332, 334, 340, 350, 352, 356) may provide some form of visual indication to show which robotic arm(s) (20, 32) is/are being controlled via one or more corresponding user input assemblies (220).

Indicators (330, 332, 334, 340, 350, 352, 356) may also provide a visual indication indicating an operational state of the corresponding instrument (14). For instance, if one indicator (330, 332, 334, 340, 350, 352, 356) is associated with an electrosurgical instrument that is capable of toggling between a cutting mode and a coagulation mode, that indicator (330, 332, 334, 340, 350, 352, 356) may visually indicate whether that electrosurgical instrument is in the cutting mode or the coagulation mode. In some such versions, the operational states may be stacked (e.g., top to bottom) corresponding to an appropriate pedal (242) on pedalboard (240). For instance, a certain set of pedals (242) may be operable to select an operational state of an instrument (14). When one of pedals (242) is pressed (e.g., a left-side pedal (242)), the instrument (14) that is under the control of one of user input assemblies (220) (e.g., the left user input assembly (220)) may be activated with the actionable state being represented in a corresponding tab (not shown) of the user interface and the bottom stacked actionable state as indicated via a corresponding one of indicators (330, 332, 334, 340, 350, 352, 356). By way of further example only, display (300) may be configured and operable in accordance with at least some of the teachings of U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety.

### III. EXAMPLE OF MODE SELECTION FOR CLIP APPLIER

In some scenarios, an operator may actuate one or more of activation arms (224), either partially or fully. For instance, the operator may actuate one or more of activation arms (224) while moving the entirety of user input assembly (220) to reposition and/or re-orient a corresponding robotic arm (20, 32). Such actuation of activation arms (224) may be intentional or inadvertent. Such actuation of activation arms (224) may result in full or partial activation of end effector (84). In scenarios where end effector (84) is a clip applying end effector like end effector (100) of FIGS. 7A-7C, and a ligation clip (150) is disposed in end effector (100), the full or partial activation of end effector (100) may result in corresponding full or partial closure of ligation clip (150).

In the event that end effector (100) is fully activated (e.g., to reach the fully closed state shown in FIG. 7C) while end effector (100) is en route to the targeted anatomical structure, the activation of end effector (100) may cause full closure of ligation clip (150); and latching features of ligation clip (150) may maintain ligation clip (150) in the closed state. This closure of ligation clip (150) may be an unintended result of intentional or unintentional actuation of activation arms (224) before end effector (100) has reached the targeted anatomical structure, such that ligation clip (150) may have prematurely reached the closed state. Once end effector (100) reaches the targeted anatomical structure and the operator opens end effector (100) to position end effector (100) around the targeted anatomical structure, the prematurely-closed and locked ligation clip (150) may fall from end effector (100), and thus end effector (100) will no longer have a ligation clip (150) to apply to the targeted anatomical structure. Similarly, undesirable results may be realized in some scenarios where ligation clip (150) has a malleable feature, a ratcheting feature, or some other feature that may maintain ligation clip (150) in a partially closed state. In such scenarios, if end effector (100) is partially activated (e.g., to reach the partially closed state shown in FIG. 7B or some other state of partial closure between the fully open state of FIG. 7A or the fully closed state of FIG. 7C) while end effector (100) is en route to the targeted anatomical structure, the partial activation of end effector (100) may cause partial closure of ligation clip (150). The malleable feature, ratcheting feature, or other feature of ligation clip (150) may maintain ligation clip (150) in the partially closed state. Once end effector (100) reaches the targeted anatomical structure and the operator opens end effector (100) to position end effector (100) around the targeted anatomical structure, the prematurely-partially-closed ligation clip (150) may fall from end effector (100), and thus end effector (100) will no longer have a ligation clip (150) to apply to the targeted anatomical structure.

In view of the foregoing, it may be desirable to provide a feature that prevents end effector (100) from closing jaws (110, 120) to a point where ligation clip (150) undesirably reaches a fully closed state (or certain state of partial closure). The following provides an example of how such functionality may be provided in connection with a robotic surgical system (10, 28). While the example described herein relates to an end effector (100) and ligation clip (150) as shown in FIGS. 7A-7C, the following teachings may be readily applied to various other kinds of end effectors and ligation clips. Moreover, the following teachings may be readily applied to other kinds of end effectors that do not necessarily apply ligation clips, such as other end effectors where premature full or partial activation of the end effector is undesirable. Similarly, while the example described herein relates to user input assemblies (220) shown in FIGS. 8 and 9A-9B, the following teachings may be readily applied to various other kinds of user input features.

To avoid premature full closure (and, in some cases, partial closure) of ligation clip (150), a control module of robotic system (10, 28), such as processor (252), may provide selection between two different modes, including a safe mode and a fire mode. While in the safe mode, the control module (e.g., processor (252), etc.) may fully prevent actuation of activation arms (224) from causing any degree of closure of jaws (110, 120). In other words, while the control module is in the safe mode, jaws (110, 120) may be non-responsive to any actuation of activation arms (224). In some other variations, while in the safe mode, the control module may permit some degree of closure of jaws (110, 120) in response to actuation of activation arms (224); yet still restrict the closure angle (Θ) that may be achieved by jaws (110, 120). In other words, if the operator inadvertently or intentionally actuates activation arms (224) while in safe mode, jaws (110, 120) may correspondingly close up to the restricted closure angle (Θ); yet fail to close any further past that angle (Θ) even if the operator fully actuates activation arms (224). In some cases, the operator may wish to partially close jaws (110, 120) to facilitate movement of end effector (100) (e.g., through a narrow anatomical space) without fully closing ligation clip (150); or for some other reason. Some versions may thus permit the operator to achieve such intentional partial closure of jaws (110, 120), up to the restricted closure angle (Θ). While in the fire mode, the control module may allow jaws (110, 120) reach the fully closed state, such that the closure angle (Θ) of jaws (110, 120) is not restricted in the fire mode.

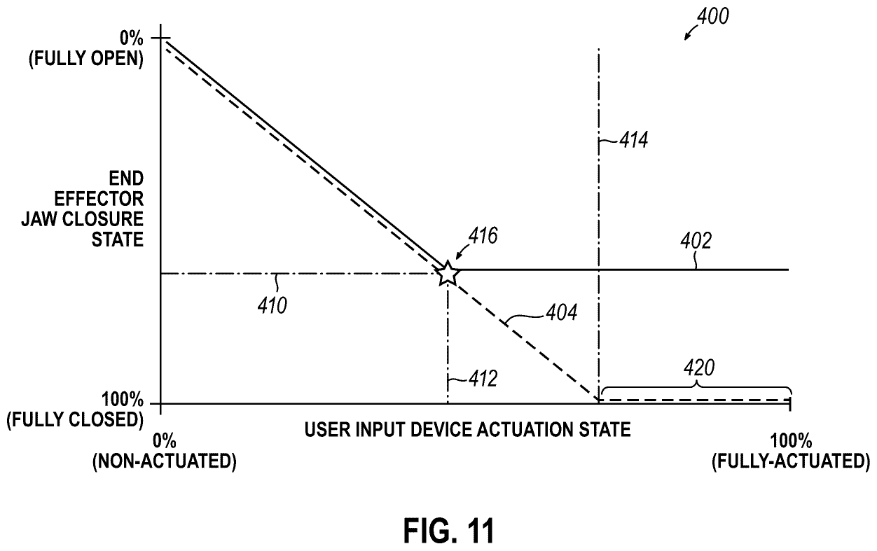

FIG. 11 shows a graph (400) with two plots (402, 404). Plot (402) is shown in solid line format while plot (404) is shown in broken line format. Plot (402) represents an example of a relationship between the state of jaws (110, 120) (y-axis) and the state of activation arms (224) (x-axis) in the safe mode. Plot (402) begins in a state where jaws (110, 120) are fully open and activation arms (224) are non-actuated. As can be seen, plot (402) includes an inflection point (416) where further actuation of activation arms (224) ceases to cause further closure of jaws (110, 120). In other words, as the operator actuates activation arms (224) (e.g., inadvertently), jaws (110, 120) will partially close up until the state of jaws (110, 120) reaches inflection point (416). Inflection point (416) is located at an intersection of lines (410, 412). Line (410) represents the value of the maximum tolerable degree to which jaws (110, 120) may be partially closed. By way of example only, this maximum tolerable degree of partial jaw (110, 120) closure may be approximately 50%, which may be associated with a closure angle (Θ) of approximately 60 degrees, a closure angle (Θ) of approximately 90 degrees, or some other predetermined permissible closure angle (Θ).

In versions where jaws (110, 120) are permitted to close up to a predetermined closure angle (Θ), this predetermined permissible closure angle (Θ) may be selected based on characteristics of ligation clip (150). For instance, ligation clip (150) may be configured such that ligation clip (150) will not plastically deform before jaws (110, 120) reach the predetermined permissible closure angle (Θ); yet ligation clip (150) will plastically deform after jaws (110, 120) surpass the predetermined permissible closure angle (Θ). Any other suitable criteria may be used to select the predetermined permissible closure angle (Θ) for the safe mode.

In scenarios where jaws (110, 120) are prevented from closing at all in the safe mode, the maximum tolerable degree of partial jaw (110, 120) closure is 0%, which is associated with the first angle (Θ1) described above with reference to FIG. 7A. Alternatively, any other suitable closure angle (Θ) may be used to provide any other suitable maximum tolerable degree of partial jaw (110, 120) closure. Line (412) represents the degree to which activation arms (224) are actuated when jaws (110, 120) reach the maximum tolerable degree of closure (410) in the safe mode.

Plot (404) represents an example of a relationship between the state of jaws (110, 120) (y-axis) and the state of activation arms (224) (x-axis) in the fire mode. Plot (404) also begins in a state where jaws (110, 120) are fully open and activation arms (224) are non-actuated. As can be seen from plot (404), when in the fire mode, jaws (110, 120) may continue toward full closure, proceeding past inflection point (416) associated with plot (402), as the operator actuates activation arms (224). In this example, line (414) indicates a point at which jaws (110, 120) reach a fully closed state, before activation arms (224) reach a fully actuated state. In other words, activation arms (224) reach a fully closed state when activation arms (224) reach a certain degree of partial actuation, with that certain degree of partial actuation being indicated by line (414). By way of example only, this certain degree of partial actuation may be approximately 90%. To the extent that the operator continues to actuate activation arms (224) past this certain degree of partial actuation associated with line (414) and full closure of jaws (110, 120), this further actuation of activation arms (224) may provide an increasing clamping force via closed jaws (110, 120). Bracket (420) indicates this range of further actuation of activation arms (224) that is associated with providing an increasing clamping force via closed jaws (110, 120). In some versions, user input assembly (220) includes a detent feature and/or other feature that provides tactile and/or audible feedback indicating when activation arms (224) have reached the degree of partial actuation associated with line (414) and full closure of jaws (110, 120). Such feedback features are optional and therefore may be omitted.

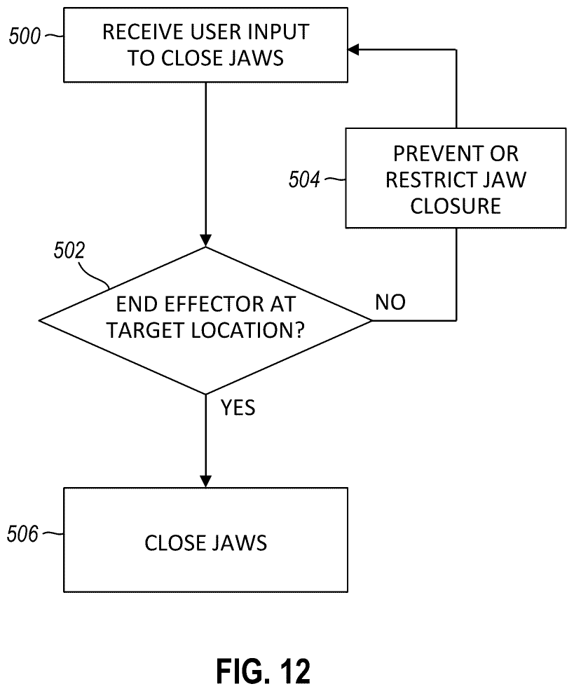

In some versions, the operator may manually toggle between the safe mode and fire mode. Such toggling of modes may be accomplished via a pedal (242) of pedalboard (240). In some other versions, a control module (e.g., processor (252), etc.) may automatically toggle between safe mode and fire mode, as will be described in greater detail below in connection with FIG. 12. In versions where the mode is toggled manually via a pedal (242), display (300) may show the activation of the safe/fire mode toggling pedal (242) via a corresponding graphical representation (322). In some versions where the mode is toggled manually via a pedal (242), the operator may simply tap pedal (242) to toggle between the modes. As another variation, the operator may need to depress pedal (242) for a certain period of time (e.g., three seconds, etc.) to toggle between the modes. In some other versions where the mode is toggled manually via a pedal (242), the control module may remain in safe mode by default (i.e., when the operator is not actuating foot pedal (242)); then switch over to fire mode while the operator actuates foot pedal (242). Once the operator releases pedal (242), the control module may automatically switch back to safe mode. In lieu of switch between modes via a pedal (242), the operator may activate a touchscreen or other user input feature to switch between the safe mode and the fire mode. Some variations may also allow the operator to define whether the control module should be in the safe mode by default, the fire mode by default, or some other mode by default. Similarly, some variations may allow the operator to define whether the control module should toggle between the safe mode and the fire mode in response to activation of a pedal (242), in response to a touchscreen or other user input feature, or in response to some other condition(s).

In addition, or in the alternative, and regardless of whether the safe and fire modes are toggled manually or automatically, the safe/fire mode state may be indicated via one or more of indicators (330, 332, 334, 340, 350, 352, 356) (e.g., via the indicator (330, 332, 334, 340, 350, 352, 356) representing the instrument (14) having end effector (100)). In addition, or in the alternative, and again regardless of whether the safe and fire modes are toggled manually or automatically, display (300) may otherwise provide some form of visual feedback indicating whether safe mode is in place or fire mode is in place.

FIG. 12 shows a workflow that may be executed via a control module (e.g., processor (252), etc.) to provide an automated version of the safe mode. As shown in block (500), processor (252) may first receive a user input to close jaws (110, 120). This user input may come in the form of intentional or inadvertent actuation of one or more of activation arms (224). After receiving this user input, the control module may determine whether end effector (100) is within sufficient proximity to the targeted anatomical structure, as shown in block (502). This may be performed based on signals from one or more position sensors in end effector (100), based on signals from one or more position sensors in another portion of instrument (14), based on signals from one or more position sensors in instrument driver (24, 66) and/or other components of robotic arm (20, 32), based on tracked kinematics associated with movement of robotic arm (20, 32), and/or based on any other source(s) of data. In the event that the control module determines that end effector (100) is not within sufficient proximity to the targeted anatomical structure, the control module may automatically provide the safe mode, such that closure of jaws (110, 120) is prevented or at least restricted despite actuation of one or more of activation arms (224), as shown in block (504). In the event that the control module determines that end effector (100) is within sufficient proximity to the targeted anatomical structure, the control module may allow full closure of jaws (110, 120) in response to actuation of one or more of activation arms (224), as shown in block (506).

As another variation, the process shown in FIG. 12 may be executed when the operator depresses a pedal (242) associated with transitioning from safe mode to fire mode. In such versions, the control module may remain in safe mode regardless of the position of end effector (100) in three dimensional space when one or more of activation arms (224) is/are actuated, such that the process shown in FIG. 12 is not even triggered unless and until the operator depresses a pedal (242) associated with transitioning from safe mode to fire mode. In some such versions, the process shown in FIG. 12 will only be executed while the operator maintains actuation of the corresponding pedal (242). Thus, control module may provide a hybrid of manual and automatic activation to transition from the safe mode to the fire mode.

In some scenarios where a pedal (242) must remain depressed in order to transition from the safe mode to the fire mode, there may be a case where the operator is at least partially actuating one or more of activation arms (224) while the pedal (242) is in a non-actuated state; and then the operator actuates pedal (242) while the one or more activation arms (224) are at least partially actuated. In some such scenarios, the control module may prevent such actuation of pedal (242) from providing a transition to the fire mode. In other words, the control module may require the operator to release activation arms (224) and then re-actuate activation arms (224) while pedal (242) is actuated, in order to achieve full jaw (110, 120) closure in a fire mode. This may prevent closure of jaws (110, 120) in response to actuation of a pedal (242), such that jaws (110, 120) will only close in response to a “fresh” actuation of activation arms (224). However, this approach is just one option and other versions may not require activation arms (224) to be fully released in the event that pedal (242) is only depressed after activation arms (224) have been at least partially actuated. For instance, in some scenarios, if the operator actuates pedal (242) while activation arms (224) are partially actuated, but before activation arms (224) have reached inflection point (416), the control module will allow such actuation of pedal (242) to cause a transition from the safe mode to the fire mode. However, if activation arms (224) have passed inflection point (416) by the time

After jaws (110, 120) have been fully closed, the ligation clip (150) has been fully closed and deployed at the targeted anatomical structure, jaws (110, 120) may be re-opened and taken away from the targeted anatomical structure. The control module may remain in the fire mode until jaws (110, 120) reach a certain degree of opening. In some scenarios, this certain degree of opening may be the same state associated with line (410) shown in FIG. 11 and described above. Once jaws (110, 120) reach this certain degree of opening, the control module may automatically toggle back to the safe mode. Alternatively, any other suitable algorithm may be applied after jaws (110, 120) have been fully closed and the ligation clip (150) has been fully closed and deployed at the targeted anatomical structure.

In view of the foregoing, a safe mode may provide an effective lockout that prevents undesirable premature closure of a ligation clip (150), though in some cases a partial closure of ligation clip (150) may be permitted in the safe mode. Such prevention of undesirable premature closure of a ligation clip (150) may prevent ligation clip (150) from undesirably falling out of end effector (100) before end effector (100) reaches the targeted anatomical structure. Similarly, such prevention of undesirable premature closure of a ligation clip (150) may avoid a scenario where ligation clip (150) is unable to fully encompass the targeted anatomical structure, even if the ligation clip (150) remained in end effector during the entire range of travel to the targeted anatomical structure.

### IV. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An assembly comprising: (a) an instrument, the instrument including: (i) a shaft assembly, and (ii) an end effector at a distal end of the shaft assembly, the end effector being operable to transition between a fully open configuration and a fully closed configuration; (b) a first user input, the first user input being operable to generate an activation signal; and (c) a control module in communication with the instrument, the control module further being in communication with the first user input, the control module being configured to transition between a safe operating mode and a fire operating mode, wherein: (i) in the safe operating mode, the control module is configured to prevent or restrict closure of the end effector, and (ii) in the fire operating mode, the control module is configured to generate a power drive signal that drives closure of the end effector to the fully closed configuration.

### Example 2

The apparatus of Example 1, the end effector being configured to receive a clip, the end effector being operable to drive closure of the clip as the end effector transitions from the fully open configuration to the fully closed configuration.

### Example 3

The apparatus of Example 2, further comprising the clip, the clip being received in the end effector, the end effector being operable to drive closure of the clip as the end effector transitions from the fully open configuration to the fully closed configuration.

### Example 4

The apparatus of Example 3, wherein the clip is configured to transition from a fully open state to a fully closed state as the end effector transitions from the fully open configuration to the fully closed configuration.

### Example 5

The apparatus of Example 4, wherein the end effector is operable to reach a predetermined partially closed configuration between the fully open configuration and the fully closed configuration, the clip being configured to reach a partially closed state between the fully open state and the fully closed state in response to the end effector reaching the predetermined partially closed configuration, and wherein: (i) in the safe operating mode, the control module is configured to allow closure of the end effector up to the predetermined partially closed position, and (ii) in the safe operating mode, the control module is configured to prevent closure of the end effector beyond the predetermined partially closed position.

### Example 6

The apparatus of Example 5, wherein the clip is capable of returning from the partially closed state to the fully open state after reaching the partially closed state.

### Example 7

The apparatus of Example 6, wherein the clip is unable to return from the partially closed state to the fully open state after surpassing the partially closed state.

### Example 8

The apparatus of Example 7, wherein the clip is configured to plastically deform after surpassing the partially closed state.

### Example 9

The apparatus of Example 7, wherein the clip includes a malleable feature preventing the clip from returning to the fully open state after surpassing the partially closed state.

### Example 10

The apparatus of any of Examples 1 through 9, further comprising a robotic arm, the instrument being coupled with the robotic arm.

### Example 11

The apparatus of Example 10, further comprising a table, the table being configured to support a patient, the robotic arm being coupled with the table.

### Example 12

The apparatus of any of Examples 1 through 11, wherein the first user input comprises one or more pivoting activation arms, the one or more pivoting activation arms being pivotable to generate the activation signal.

### Example 13

The apparatus of any of Examples 1 through 12, further comprising a console, the first user input being integrated into the console, the console further including a viewing assembly.

### Example 14

The apparatus of Example 13, the viewing assembly being operable to display an endoscopic view of the end effector in a surgical field.

### Example 15

The apparatus of Example 14, the viewing assembly further being operable to generate a visual indicator, the visual indicator indicating whether the control module is in the safe mode or the fire mode.

### Example 16

The apparatus of any of Examples 1 through 15, further comprising a second user input, the control module being configured to transition from the safe mode to the fire mode in response to a mode transition signal from the second user input.

### Example 17

The apparatus of Example 16, the second user input comprising a foot-actuated pedal.

### Example 18

The apparatus of any of Examples 16 through 17, the control module being configured to transition from the safe mode to the fire mode in response to the second user input being actuated for a predetermined period of time.

### Example 19

The apparatus of Example 18, the control module being configured to remain in the fire mode after the second user input has been actuated for the predetermined period of time.

### Example 20

The apparatus of Example 17, the control module being configured to transition from the safe mode to the fire mode in response to the second user input being actuated, the control module being further configured to transition from the fire mode back to the safe mode in response to cessation of actuation of the second user input.

### Example 21

The apparatus of any of Examples 1 through 20, the control module being further configured to identify a real-time position of the end effector, the control module being configured to transition from the safe operating mode to the fire operating mode based at least in part on the real-time position of the end effector.

### Example 22

The apparatus of any of Examples 1 through 21, further comprising a tower, the control module being incorporated into the tower.

### Example 23

The apparatus of any of Examples 1 through 22, the end effector including: (A) a first jaw, and (B) a second jaw, the first and second jaws being operable to transition between a fully open position and a fully open position, thereby providing the end effector in the fully open configuration and the fully closed configuration, respectively.

### Example 24

An apparatus, comprising: (a) a first user input, the first user input being operable to generate an activation signal; and (b) a control module in communication with an instrument, the control module further being in communication with the first user input, the control module being configured to transition between a safe operating mode and a fire operating mode, wherein: (i) in the safe operating mode, the control module is configured to prevent or restrict closure of the instrument, and (ii) in the fire operating mode, the control module is configured to generate a power drive signal that drives closure of the instrument to the fully closed position.

### Example 25

The apparatus of Example 24, further comprising the instrument, the instrument including: (i) a shaft assembly, and (ii) an end effector at a distal end of the shaft assembly, the end effector including: (A) a first jaw, and (B) a second jaw, first and second jaws being operable to transition between the fully open position and the fully closed position; the control module being in communication with the instrument, wherein: (i) in the safe operating mode, the control module is configured to prevent or restrict closure of the first and second jaws in response to the activation signal from the first user input, and (ii) in the fire operating mode, the control module is configured to generate a power drive signal that drives closure of the first and second jaws to the fully closed position.

### Example 26

A method comprising: (a) receiving an activation signal from a first user input; (b) determining whether a control module is in a safe mode or a fire mode; (c) if the control module is in the safe mode, preventing or restricting closure of jaws of an end effector of an instrument in response to the activation signal; and (d) if the control module is in the fire mode, allowing full closure of the jaws of the end effector in response to the activation signal.

### Example 27

The method of Example 26, the user input including one or more pivoting activation arms, the activation signal being generated by pivotal movement of the one or more pivoting activation arms.

### Example 28

The method of any of Examples 26 through 27, the jaws of the end effector being engaged with a clip.

### Example 29

The method of Example 28, the clip being configured to return from a partially closed state to a fully open state after reaching the partially closed state, the clip being unable to return from the partially closed state to the fully open state after surpassing the partially closed state.

### Example 30

The method of Example 29, if the control module is in the safe mode, preventing the jaws of the end effector from driving the clip past the partially closed state.

### Example 31

The method of Example 30, if the control module is in the safe mode, permitting the jaws of the end effector to drive the clip up to the partially closed state in response to the activation signal.

### Example 32

The method of any of Examples 26 through 31, further comprising driving an indicator on a display, the indicator indicating whether the control module is in the safe mode or the fire mode.

### Example 33

The method of Example 32, the display further including an endoscopic image of the end effector in a surgical field.

### Example 34

The method of any of Examples 26 through 33, further comprising: a) receiving a mode transition signal from a second input; and (b) transitioning the control module from the safe mode to the fire mode in response to at least the mode transition signal from the second input.

### Example 35

The method of Example 34, the mode transition signal from the second input being generated in response to actuation of the second input for a predetermined period of time.

### Example 36

The method of Example 34, the mode transition signal from the second input being generated while the second input is being actuated, the mode transition signal from the second input ceasing when the second input is no longer being actuated.

### Example 37

The method of Example 36, further comprising transitioning the control module from the fire mode to the safe mode in response to the mode transition signal from the second input ceasing.

### Example 38

The method of any of Examples 26 through 37, further comprising: (a) receiving an end-effector position signal indicating a real-time position of the end effector; and (b) transitioning the control module from the safe mode to the fire mode in response to at least the end-effector position signal indicating the real-time position of the end effector.

### Example 39

The method of Example 38, the real-time position of the end effector being within a certain distance of a targeted anatomical structure.

### Example 40

A processor-readable medium including contents that are configured to cause a processor to process data by performing the method of any of Examples 26 through 39.

### Example 41

A non-transitory computer readable medium storing instructions operable to, when executed by a processor, cause a robotic surgical system to perform a set of tasks comprising: (a) receiving an activation signal from a first user input; (b) determining whether a control module is in a safe mode or a fire mode; (c) if the control module is in the safe mode, preventing or restricting closure of jaws of an end effector of an instrument; and (d) if the control module is in the fire mode, allowing full closure of the jaws of the end effector.

### V. MISCELLANEOUS

Some versions of the examples described herein may be implemented using a processor, which may be part of a computer system and communicate with a number of peripheral devices via bus subsystem. Versions of the examples described herein that are implemented using a computer system may be implemented using a general-purpose computer that is programmed to perform the methods described herein. Alternatively, versions of the examples described herein that are implemented using a computer system may be implemented using a specific-purpose computer that is constructed with hardware arranged to perform the methods described herein. Versions of the examples described herein may also be implemented using a combination of at least one general-purpose computer and at least one specific-purpose computer.

In versions implemented using a computer system, each processor may include a central processing unit (CPU) of a computer system, a microprocessor, an application-specific integrated circuit (ASIC), other kinds of hardware components, and combinations thereof. A computer system may include more than one type of processor. The peripheral devices of a computer system may include a storage subsystem including, for example, memory devices and a file storage subsystem, user interface input devices, user interface output devices, and a network interface subsystem. The input and output devices may allow user interaction with the computer system. The network interface subsystem may provide an interface to outside networks, including an interface to corresponding interface devices in other computer systems. User interface input devices may include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

In versions implemented using a computer system, a storage subsystem may store programming and data constructs that provide the functionality of some or all of the modules and methods described herein. These software modules may be generally executed by the processor of the computer system alone or in combination with other processors. Memory used in the storage subsystem may include a number of memories including a main random-access memory (RAM) for storage of instructions and data during program execution and a read only memory (ROM) in which fixed instructions are stored. A file storage subsystem may provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem in the storage subsystem, or in other machines accessible by the processor.

In versions implemented using a computer system, the computer system itself may be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the example of the computer system described herein is intended only as a specific example for purposes of illustrating the technology disclosed. Many other configurations of a computer system are possible having more or fewer components than the computer system described herein.

As an article of manufacture, rather than a method, a non-transitory computer readable medium (CRM) may be loaded with program instructions executable by a processor. The program instructions when executed, implement one or more of the computer-implemented methods described above. Alternatively, the program instructions may be loaded on a non-transitory CRM and, when combined with appropriate hardware, become a component of one or more of the computer-implemented systems that practice the methods disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof is placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. An apparatus comprising:
(a) an instrument, the instrument including:
(i) a shaft assembly, and
(ii) an end effector at a distal end of the shaft assembly, the end effector being operable to transition between a fully open configuration and a fully closed configuration;
(b) a first user input, the first user input being operable to generate an activation signal; and
(c) a control module in communication with the instrument, the control module further being in communication with the first user input, the control module being configured to transition between a safe operating mode and a fire operating mode, wherein:
(i) in the safe operating mode, the control module is configured to prevent or restrict closure of the end effector, and
(ii) in the fire operating mode, the control module is configured to generate a power drive signal that drives closure of the end effector to the fully closed configuration.

2. The apparatus of claim 1, the end effector being configured to receive a clip, the end effector being operable to drive closure of the clip as the end effector transitions from the fully open configuration to the fully closed configuration.

3. The apparatus of claim 2, further comprising the clip, the clip being received in the end effector, wherein the clip is configured to transition from a fully open state to a fully closed state as the end effector transitions from the fully open configuration to the fully closed configuration.

4. The apparatus of claim 3, wherein the end effector is operable to reach a predetermined partially closed configuration between the fully open configuration and the fully closed configuration, the clip being configured to reach a partially closed state between the fully open state and the fully closed state in response to the end effector reaching the predetermined partially closed configuration, and wherein:
(i) in the safe operating mode, the control module is configured to allow closure of the end effector up to the predetermined partially closed configuration, and
(ii) in the safe operating mode, the control module is configured to prevent closure of the end effector beyond the predetermined partially closed configuration.

5. The apparatus of claim 4, wherein the clip is capable of returning from the partially closed state to the fully open state after reaching the partially closed state, and wherein the clip is unable to return from the partially closed state to the fully open state after surpassing the partially closed state.

6. The apparatus of claim 5, wherein the clip is configured to plastically deform after surpassing the partially closed state.

7. The apparatus of claim 5, wherein the clip includes a malleable feature preventing the clip from returning to the fully open state after surpassing the partially closed state.

8. The apparatus of claim 1, further comprising a robotic arm, the instrument being coupled with the robotic arm.

9. The apparatus of claim 1, wherein the first user input comprises one or more pivoting activation arms, the one or more pivoting activation arms being pivotable to generate the activation signal.

10. The apparatus of claim 1, further comprising a console, the first user input being integrated into the console, the console further including a viewing assembly, the viewing assembly being operable to display an endoscopic view of the end effector in a surgical field, the viewing assembly further being operable to generate a visual indicator, the visual indicator indicating whether the control module is in the safe mode or the fire mode.

11. The apparatus of claim 1, further comprising a second user input, the control module being configured to transition from the safe mode to the fire mode in response to a mode transition signal from the second user input.

12. The apparatus of claim 11, the second user input comprising a foot-actuated pedal.

13. The apparatus of claim 11, the control module being configured to transition from the safe mode to the fire mode in response to the second user input being actuated for a predetermined period of time.

14. The apparatus of claim 13, the control module being configured to remain in the fire mode after the second user input has been actuated for the predetermined period of time.

15. The apparatus of claim 12, the control module being configured to transition from the safe mode to the fire mode in response to the second user input being actuated, the control module being further configured to transition from the fire mode back to the safe mode in response to cessation of actuation of the second user input.

16. The apparatus of claim 1, the control module being further configured to identify a real-time position of the end effector, the control module being configured to transition from the safe operating mode to the fire operating mode based at least in part on the real-time position of the end effector.

17. The apparatus of claim 1, further comprising a tower, the control module being incorporated into the tower.

18. The apparatus of claim 1, the end effector including:
(A) a first jaw, and
(B) a second jaw, the first and second jaws being operable to transition between a fully open position and a fully open position, thereby providing the end effector in the fully open configuration and the fully closed configuration, respectively.

19. An apparatus, comprising:
(a) a first user input, the first user input being operable to generate an activation signal; and
(b) a control module in communication with an instrument, the control module further being in communication with the first user input, the control module being configured to transition between a safe operating mode and a fire operating mode, wherein:
(i) in the safe operating mode, the control module is configured to prevent or restrict closure of the instrument, and
(ii) in the fire operating mode, the control module is configured to generate a power drive signal that drives closure of the instrument to a fully closed position.

20. A method comprising:
(a) receiving an activation signal from a first user input;
(b) determining whether a control module is in a safe mode or a fire mode;
(c) if the control module is in the safe mode, preventing or restricting closure of jaws of an end effector of an instrument in response to the activation signal; and
(d) if the control module is in the fire mode, allowing full closure of the jaws of the end effector in response to the activation signal.
